(19) United States
(12) Patent Application Publication
(10) Pub. No.: US 2025/0082001 A1
(43) Pub. Date: Mar. 13, 2025

(54) IMMUNOGLOBULIN ENRICHED MILK FRACTION

(71) Applicant: FrieslandCampina Nederland B.V., Amersfoort (NL)

(72) Inventors: Weiwei Li, Wageningen (NL); Chris Thérèse Emilienne Dotremont, Wageningen (NL); Christine Counet, Wageningen (NL)

(21) Appl. No.: 18/720,565

(22) PCT Filed: Dec. 14, 2022
(86) PCT No.: PCT/EP2022/085821

§ 371 Date: Jun. 14, 2024

(30) Foreign Application Priority Data
Dec. 15, 2021 (EP) 21214662.5

(51) Int. Cl. A23L 33/00; A23C 9/142; A23C 9/146; A23C 9/15; A23C 19/05; C07K 16/04

(52) U.S. Cl. CPC A23L 33/40; A23C 9/1422; A23C 9/1465; A23C 9/1512; A23C 19/05; C07K 16/04

(57) ABSTRACT

Process for obtaining a milk fraction enriched in immunoglobulins, preferably enriched in IgA and IgM, comprising the steps of (a) subjecting skimmed milk to microfiltration, resulting in an MF retentate rich in micellar casein and an MF permeate rich in whey proteins, (b) subjecting the MF retentate to a casein precipitation or cheese-making process, thereby creating a casein-rich fraction and a whey fraction, (c) subjecting the whey fraction obtained in step b) to microfiltration and/or anion exchange chromatography, thereby obtaining a milk fraction enriched in immunoglobulins.

The present invention relates to a milk fraction with a high immunoglobulin content, its production and use.

Milk provides the sole source of nutrition for mammalian offspring until they are able to digest food from other sources. Colostrum and milk of all lactating animals contain immunoglobulins (Ig's), which provide the offspring immunological protection against microbial pathogens and toxins and protect the mammary gland against infections. The major classes of immunoglobulins in bovine and human milk are IgG, IgA, and IgM, which differ in structure and biological activity. IgG can be subdivided in IgG₁ and IgG₂; IgA can be subdivided in in IgA₁ and IgA₂.

Immunoglobulins differ in molecular mass. The monomeric IgG has a mass of about 150 kDa (the precise weight depends on the originating animal), dimeric IgA about 400 kDa, and IgM about 900 kDa, which is significantly higher than the weight of the main whey proteins (about 14-18 kDa) and significantly smaller than the weight of the casein micelles present in milk.

In human milk, the major Ig is IgA. Human breast milk contains—based on total Ig-content—about 85-90 wt % IgA, about 2-3 wt % IgG, and about 8-10 wt % IgM (J. A. Cakebread et al., J. Agric. Food Chem., 63 (2015) 7311-7316). The total Ig concentration in human breast milk is about 1.5 times the Ig content of bovine milk (J. A. van Neerven et al., J. Allergy Clin. Immunol., October 2012, 853-858).

In bovine milk, the major Ig is IgG. Mature bovine milk contains—based on total Ig content—about 80 wt % IgG (the far majority being IgG₁), about 10 wt % IgM, and about 10 wt % IgA.

The Ig-content of bovine colostrum is much higher than that of mature bovine milk: 70-80% of the total protein content of colostrum are Ig's, whereas in mature bovine milk they only provide for 1-2 wt % of the total protein content. The IgG/IgA ratio in colostrum is even higher than in mature bovine milk.

Infant formula is conventionally . . . prepared by combining at least one source of whey protein, at least a source of milk (casein) proteins, at least one source of lipids, at least one carbohydrate source, vitamins, and minerals. Bovine milk is one of the applied sources for these proteins, carbohydrates, lipids, and vitamins.

There is a continuing desire to produce infant formula which resembles human breast milk as closely as possible. Hence, there is a desire to increase the active immunoglobulin content and the IgA content of infant formula in order to reach that goal. One of the objects of the present invention is therefore the provision of a milk fraction having a high ratio of IgA relative to IgG; so that addition of this milk fraction to infant formula will meet the above desire.

An efficient manner of obtaining whey proteins involves microfiltration of skimmed milk into a casein-rich retentate and a whey protein-rich permeate.

The whey protein-rich permeate (i.e. the serum protein fraction) resulting from the microfiltration can be used to make serum protein concentrate (SPC) by subjecting said permeate to ultrafiltration in order to remove lactose, ash, and water, followed by (spray) drying.

From the casein-rich retentate, micellar casein isolate (MCI) can be obtained by subjecting the retentate to evaporation and (spray) drying. This MCI can be used for, e.g., the production of soft cheeses like mozzarella and feta cheese. The whey fraction of this cheese-making process is generally regarded as waste, despite the fact that it contains significant amounts of Ig's. Another object of the present invention is therefore to valorize the Ig's within this whey fraction.

During microfiltration, most of the IgG ends up in the serum protein fraction. The casein fraction, on the other hand, contains most of the IgA and IgM, in addition to a non-negligible portion of IgG.

The present invention relates to a method for valorizing the immunoglobulins that remain in the casein-rich MF retentate.

More in particular, it relates to a milk fraction comprising a content of immunoglobulins—defined as the total content of IgG+IgA+IgM—relative to total protein content, in the range 10 to 40 wt % and a weight ratio (IgA+IgM)/IgG in the range 25-60 wt %, preferably 45-60 wt %.

The invention also relates to a process for obtaining an Ig-enriched milk fraction, preferably enriched in IgA and IgM, comprising the steps of:
a) subjecting skimmed milk to microfiltration, resulting in an MF retentate rich in micellar casein and an MF permeate rich in whey proteins,
b) subjecting the MF retentate to a casein-precipitation and/or cheese-making process, thereby creating a casein-rich fraction and a whey fraction,
c) subjecting the whey fraction obtained in step b) to microfiltration and/or anion exchange chromatography, thereby obtaining a milk fraction enriched in immunoglobulins.

The first step in the process of the present invention involves microfiltration of skimmed milk. The term “skimmed milk” refers to animal milk, preferably bovine milk, which has been skimmed and which may optionally have been pre-treated—e.g. pasteurized, diluted, ultrafiltered, concentrated, demineralized, and/or subjected to a carbohydrate level reduction—provided that the original percentages of casein to whey protein have remained substantially unaltered.

The skimmed milk is subjected to microfiltration in order to obtain a casein-rich retentate and a whey protein-rich permeate. This is a well-known process.

Various types of microfiltration membranes can be used, including spiral wound membranes, ceramic membranes, and hollow fiber membranes. The microfiltration membrane should be permeable to water, minerals, and lactose, it should be at least partially permeable to whey proteins, and should have a high retention of casein protein and fat. Suitable microfiltration membranes typically have a pore size of 0.85 to 0.5 μm, more typically 0.1 to 0.2 μm.

The microfiltration step is preferably conducted at a temperature that is either in the range 4-15° C. or 50-55° C., more preferably 10-15° C. or 50-52° C.

The cross-flow is preferably high: preferably in the range 0.10-0.25 m/s for spiral wound membranes, 1.0-2.4 m/s for hollow fiber membranes, and 5.0-7.0 m/s for ceramic membranes.

If desired, the pH of the milk can be adjusted prior to microfiltration in order to adjust the ionic charges on the molecules and the membrane. The pH is preferably in the range 5.6-6.2, more preferably 5.6-6.0, and most preferably 5.6-5.8.

The transmembrane pressure (TMP) is preferably as low as possible, more preferably in the range 0.25-1 bar.

In order to further improve the separation between immunoglobulins, diafiltration is desired.

The MF retentate is subjected to a casein-precipitation and/or a cheese-making process in order to reduce the casein content of the MF retentate.

The casein-precipitation process involves the addition of an acid to induce coagulation of casein, resulting in an acid casein fraction (a casein curd) and an acid whey fraction. The acid is preferably selected from HCl, H₂SO₄, and citric acid.

For example, 1 M H₂SO₄ may be added to skim milk, with sufficient stirring at 40-45° C., until a pH on the range 4.3-4.6 is reached. The acidified milk is stirred until curd formation is complete. The curd is then removed from the whey by means of, e.g., bag filtration or centrifugation.

The cheesemaking process preferably involves (i) addition of a fat source to the MF retentate; optionally after drying and re-suspending the MF retentate, (ii) the subsequent addition of a coagulant and an acidifier, and (iii) coagulation in order to obtain a casein-rich fraction (the cheese or cheese precursor) and a whey fraction.

Suitable fat sources are anhydrous milk fat, anhydrous milk fat fractions, butter, butter oil, cream having a fat content in the range of 30 to 80 wt %, and mixtures of two or more of these, with anhydrous milk fat and cream being the preferred fat sources.

The fat source is preferably added to the MF retentate—or a re-suspended dried retentate—at a temperature above the melting temperature of the fat source.

The target pH for the acidification is preferably in the range 4.8 to 5.7, more preferably 4.9 to 5.5. Suitable acidifiers include starter cultures (bacterial acidifiers; which convert lactose into lactic acid), acids, acidulants (such as Glucono Delta Lactone or GDL), and combinations of two or more of these. The most common starter cultures include thermophilic starters, typically starters from CSK, Chr. Hansen, or DuPont. Thermophilic starters by Chr. Hansen include frozen cultures STI-02, STI-03, STI-04, STI-06 and freeze-dried cultures STI-12, STI-13 and STI-14. Mesophilic starters may also be used.

Suitable coagulants are known in the art and include, for instance, calf rennet, fermentation-produced rennet and microbial rennet. Examples of calf rennet include Kalase produced by CSK and Naturen produced by Chr. Hansen. Examples of fermentation-produced rennet include Fromase by DSM and Milase by CSK. Examples of microbial rennets are Chy-Max by Chr. Hansen and Maxiren by DSM. Other coagulants include pepsin and various proteolytic enzymes of plant origin.

The whey fraction remaining after said casein-precipitation or cheese-making process—i.e. the acid whey fraction or cheese whey fraction, respectively—is subsequently subjected to a microfiltration step and/or an anion exchange chromatography step, thereby resulting in a milk fraction enriched in immunoglobulins.

This microfiltration step is performed with a membrane having a pore size in the range 50-100 nm or a molecular weight cut-off (MWCO) in the range 500-800 kDa.

The pore size is determined by gas-liquid porometry, also known as Capillary Flow Porometry (CFP) with perfluoroether as wetting liquid.

The MWCO is an experimentally determined parameter that represents the molecular weight of a certain solute molecule—in this case: dextran (2000 ppm aqueous solution)—that is rejected by the membrane on a level of 90%. The transmembrane pressure in this determination is about 30 psi and the dextran rejection is measured by Total Organic Carbon (TOC) determination.

A membrane with a pore size or MWCO as defined above is permeable for at least part of the IgG, but less for IgA and IgM. Hence, the resulting retentate—i.e. the milk fraction according to the invention—will be enriched in IgA and IgM, relative to IgG.

The microfiltration is preferably performed after acidification of the whey fraction to a pH in the range 4-5. The cross flow used for microfiltration is preferably 0.10-0.25 m/s for spiral wound membranes or 5-7 m/s for ceramic membranes. The applied temperature is preferably 10-15° C. The whey fraction is preferably concentrated by said microfiltration to a Volume Concentration Factor (VCF) of at least 10, and is preferably followed by at least one step of diafiltration, with equal volume of dia-water. The retentate from the microfiltration step is enriched with Ig's, especially IgA and IgM.

The anion exchange chromatography can be performed with various types of resins, including weak and strong ion exchange resins, and using various counter ions.

The terms ‘weak’ ion exchange and ‘strong’ ion exchange are generally known in the art. A strong ion exchanger will not significantly loose the charge on its matrix once the ion exchanger is equilibrated, and so a wide pH range—generally from strongly acidic pH to a strongly alkaline pH—can be used. Strong anion exchange resins are generally characterized by the presence of quaternary ammonium groups.

Weak ion exchangers have a more specific range of pH values in which they will maintain their charge, usually an acidic to about neutral pH in the case of weak anion exchange materials. Weak anion exchange groups are generally characterized by the absence of quaternary ammonium groups. Common weak anion exchange groups are tertiary amine groups.

In a preferred embodiment, a strong anion exchange resin is used, more preferably an anion exchange resin with quaternary ammonium groups (e.g., Q Sepharose Big Beads). More preferably, the anion exchange resin is the Cl⁻ form.

The anion exchange chromatography is preferably performed by first adjusting the pH of the whey fraction to 6.5-7.0. By passing through the column, the whey fraction is enriched with Ig's, especially IgA and IgM; impurity proteins such as beta-lactoglobulin, are bound to the resin and can be eluded from the column by rinsing with a buffer of low pH or high conductivity (e.g. 1 M NaCl).

It is also possible to combine the microfiltration and the anion exchange chromatography by first performing the microfiltration, adjusting the pH of the retentate to 6.5-7.0, followed by loading said retentate on the anion exchange column for further enrichment of Ig's. Such a combination allows a further enrichment of Ig's, up to about 6-8 times compared to that of the whey fraction after cheese-making or casein precipitation.

The resulting product, here referred to as “milk fraction”, has a total content of immunoglobulins, relative to total protein content, in the range 10 to 40 wt % and a weight ratio (IgA +IgM)/IgG in the range 25-60 wt %, preferably 45-60% wt %.

The total content of immunoglobulins is defined as the total content of IgG+IgA+IgM, as determined by the bovine IgG ELISA quantitation set as described by R. L. Valk-Weebera, T. Eshuis-de Ruiter, L. Dijkhuizen, and S. S. van Leeuwen, International Dairy Journal, Volume 110, November 2020, 104814). The total protein content is measured by the well-known Kjeldahl nitrogen analysis method and the application of a Kjeldahl factor of 6.38.

The immunoglobulin-enriched milk fraction according to the invention is particularly suitable for use as an ingredient in the production of a nutritional composition, in particular formula milk. The formula milk is selected from the group of infant formulas, follow-up formulas and growing-up formulas. Accordingly, the invention further relates to a nutritional composition, typically a nutritional composition for a child, such as formula milk, in particular an infant formula, a follow-up formula, or a growing-up formula.

The nutritional composition, in particular the formula milk, can be prepared by combining the immunoglobulin-enriched milk fraction with at least a lipid source, a carbohydrate source, vitamins, and minerals.

The lipid source may be any lipid or fat suitable for use in formula milk. Preferred fat sources include milk fat, safflower oil, egg yolk lipid, canola oil, olive oil, coconut oil, palm kernel oil, soybean oil, fish oil, palm oleic, high oleic sunflower oil and high oleic safflower oil, and microbial fermentation oil containing long-chain, polyunsaturated fatty acids. In one embodiment, anhydrous milk fat is used. The lipid source may also be in the form of fractions derived from these oils such as palm olein, medium chain triglycerides, and esters of fatty acids such as arachidonic acid, linoleic acid, palmitic acid, stearic acid, docosahexaeonic acid, linolenic acid, oleic acid, lauric acid, capric acid, caprylic acid, caproic acid, and the like. Small amounts of oils containing high quantities of preformed arachidonic acid and docosahexaenoic acid, such as fish oils or microbial oils, may be added. The fat source preferably has a ratio of n-6 to n-3 fatty acids of about 5:1 to about 15:1; for example about 8:1 to about 10:1. In a specific aspect, the formula milk comprises an oil mix comprising palmitic acid esterified to triacylglycerols, for example wherein the palmitic acid esterified in the sn-2 position of triacylglycerol is in the amount of from 20% to 60% by weight of total palmitic acid and palmitic acid esterified in the sn-1/sn-3 position of triacylglycerol is in the amount of from 40% to 80% by weight of total palmitic acid.

Examples of vitamins and minerals that are preferably present in formula milk are vitamin A, vitamin B1, vitamin B2, vitamin B6, vitamin B12, vitamin E, vitamin K, vitamin C, vitamin D, folic acid, inositol, niacin, biotin, pantothenic acid, choline, calcium, phosphorous, iodine, iron, magnesium, copper, zinc, manganese, chloride, potassium, sodium, selenium, chromium, molybdenum, taurine, and L-carnitine. Minerals are usually added in salt form.

Examples of carbohydrates that are preferably present in formula milk are lactose, non-digestible oligosaccharides such as galacto-oligosaccharides (GOS) and/or fructo-oligosaccharides (FOS) and human milk oligosaccharides (HMOs).

If necessary, the nutritional composition may contain emulsifiers and stabilizers such as soy lecithin, citric acid esters of mono-and di-glycerides, and the like. It may also contain other substances which may have a beneficial effect such as lactoferrin, nucleotides, nucleosides, and the like.

### EXAMPLES

### Example 1

A stream of skim milk, containing 270 ppm IgG, 30 ppm IgA, and 24 ppm IgM and having a total protein concentration of 3.9 wt % (Ig content, relative to total protein: 0.84 wt %) and a was subjected to microfiltration using a spiral wound membrane with a pose size of 0.1 μm. Microfiltration was performed at 15° C. After concentration and diafiltration, the MF retentate (MCI) was collected.

The resulting MF retentate (MCI) contained abound 500 ppm IgG, 170 ppm IgA and 100 ppm of IgM and had a total protein concentration of 14.7%. The Ig content, relative to total protein, in the MF retentate was 0.52%, i.e. lower than that of the skimmed milk, due to permeation of IgG through the membrane. The (IgA+IgM)/IgG ratio, on the other hand, increased from around 22 wt % in the skimmed milk to around 58 wt % in the MF retentate.

Next, the MF retentate was acidified to pH=4.3 using 2M H₂SO₄ at 42° C. Curd formation was allowed by letting the solution stand for about 30 minutes. The curd was then removed from the whey using a bag filter of 100 μm. The resulting whey fraction still had the same content of Ig's but the total protein content was reduced to 1.3 wt %, resulting in a total Ig content, based on total protein, of 6 wt %.

The whey fraction, having a pH of 4.3, was directly fed to a spiral wound MF (membrane area 6.7 m²) with a pore size of 500 kD. Microfiltration was performed at 15° C. A TMP of 0.5 bar was used and the cross flow was 80 l/min. The whey was concentrated to a VCF of 12 and the retentate was diafiltrated once with an equal amount of demi-water. The retentate product had a total Ig content, based on total protein, of 13 wt %. The (IgA+IgM)/IgG ratio remained 58 wt %.

### Example 2

Example 1 was repeated except that the pH of the whey fraction was adjusted to 7 and this whey fraction was then submitted to anion exchange chromatography—using a strong anion exchange resin with quaternary ammonium function groups and Cl⁻ counter ions—at room temperature, with a flow rate of 30 BV/h (bed volume per hour). Three bed volumes whey fraction were fed to the column and were collected from the outlet of the column. The collected product had a total Ig content of 20 wt %, based on total protein and no change in (IgA+IgM)/IgG ratio (58 wt %).

Afterwards, the column was eluded with a 1M NaCl solution at a flow rate of 10 BV/h in order to remove the bonded impurities; mainly beta-lactoglobulin.

### Example 3

Example 1 was repeated, except that the MF retentate obtained at the end of Example 1 was neutralized to pH 7 and submitted to anion exchange following the procedure of Example 2. The resulting product had a total Ig content, based on total protein, of 25 wt %. No change in (IgA+IgM)/IgG ratio (58 wt %) was observed.

1. Process for obtaining a milk fraction enriched in immunoglobulins, preferably enriched in IgA and IgM, comprising the steps of:
a) subjecting skimmed milk to microfiltration, resulting in an MF retentate rich in micellar casein and an MF permeate rich in whey proteins,
b) subjecting the MF retentate to a casein precipitation or cheese-making process, thereby creating a casein-rich fraction and a whey fraction,
c) subjecting the whey fraction obtained in step b) to microfiltration and/or anion exchange chromatography, thereby obtaining a milk fraction enriched in immunoglobulins.

2. Process according to claim 1 wherein the pH of the skimmed milk is adjusted to a value in the range 5.6-6.2, more preferably 5.6-6.0, and most preferably 5.6-5.8, prior to microfiltration.

3. Process according to claim 1 wherein the cheese-making process involves (i) addition of a fat source to the MF retentate, optionally after drying and re-suspending the MF retentate, (ii) subsequent addition of a coagulant and an acidifier, and (iii) coagulation in order to obtain a casein-rich fraction and a whey fraction.

4. Process according to claim 1 wherein the whey fraction obtained in step b) is acidified to a pH in the range 4-5 before being subjected to microfiltration in step c).

5. Process according to claim 1 wherein the microfiltration of step c) is performed with a membrane having a pore size in the range 50-100 nm or a molecular weight cut-off (MWCO) in the range 500-800 kDa.

6. Process according to claim 1 wherein the microfiltration of step c) is performed with a spiral wound membrane using a cross flow of 0.10-0.25 m/s or with a ceramic membrane using a cross-flow of 5-7 m/s.

7. Process according to claim 1 wherein the microfiltration of step c) is performed at a temperature in the range 10-15° C.

8. Process according to claim 1 wherein the whey fraction obtained in step b) is acidified to a pH in the range 6.5-7 before being subjected to anion exchange chromatography.

9. Milk fraction comprising a content of immunoglobulins, relative to total protein content, in the range 10 to 40 wt % and a weight ratio (IgA+IgM)/IgG in the range 25-60 wt %, preferably 45-60 wt %.

10. Nutritional composition selected from infant formula, follow-up formula, and growing-up milk comprising the milk fraction according to claim 9.

11. Process for producing the nutritional composition of claim 10 by combining the milk fraction with at least a fat source, a carbohydrate source, a whey protein source, and vitamins and minerals.
